(19) United States
(12) Patent Application Publication
(10) Pub. No.: US 2026/0192010 A1
(43) Pub. Date: Jul. 9, 2026

(54) FRAGRANCE DELIVERY DEVICE

(71) Applicant: FIRMENICH SA, Satigny (CH)

(72) Inventors: Rutger VAN SLEEUWEN, Bordentown, NJ (US); Pooja PATEL, Plainsboro, NJ (US); Huda JERRI, Westampton, NJ (US); Nicholas O'LEARY, Pennington, NJ (US)

(73) Assignee: FIRMENICH SA, Satigny (CH)

(21) Appl. No.: 19/129,724

(22) PCT Filed: Nov. 22, 2023
(86) PCT No.: PCT/EP2023/082675

§ 371 Date: May 14, 2025

Related U.S. Application Data
(60) Provisional application No. 63/384,866, filed on Nov. 23, 2022.

(30) Foreign Application Priority Data
May 4, 2023 (EP) 23171479.1

(51) Int. Cl. A61L 9/12; A61L 9/012

(52) U.S. Cl. CPC A61L 9/127; A61L 9/012; A61L 9/125; A61L 2209/134; A61L 2209/21

(57) ABSTRACT

The present disclosure relates to the field of perfumery and more precisely it concerns a device and associated consumer articles, for dispersing a liquid fragrance composition into the surrounding space via evaporation.

### CROSS REFERENCE TO RELATED APPLICATIONS

This application claims the benefit of priority of U.S. Provisional Application No. 63/384,866, filed Nov. 23, 2022, and European Application No. 23171479.1, filed May 4, 2023. The entire contents of these applications are explicitly incorporated herein by this reference.

### FIELD OF THE INVENTION

The present disclosure relates to the field of perfumery and more precisely it concerns a device and associated consumer articles, for dispensing a liquid fragrance composition into the surrounding space.

### BACKGROUND

Air care devices, typically air freshener devices, for dispensing liquid fragrance compositions into the surrounding space are known. Many air fresheners are commercially available in different formats such as reed or wick diffusers, electrical plug-in devices, aerosols, or sprays. The fragrance composition of such air fresheners may be a fragrance oil, or a mixture of several fragrance oils with or without a suitable solvent, or a colloidal solution such as a microemulsion. However, while they provide certain benefits, such as freshening the air or masking or eliminating malodors, conventional liquid air fresheners often suffer from certain disadvantages and limitations.

For example, air freshener performance may be unsatisfactory due to limited or even unacceptable fragrance performance, typically related to low evaporation rate, poor product longevity, mediocre aesthetics, limited shapes and forms, variable fragrance release, variable odor quality, and/or the need for an active power source, such as heaters, batteries, or electricity, and the use of generic, non-premium materials.

WO2020058373 discloses a device comprising a body portion and at least one active composition, which is selected from the group consisting of an active composition comprising a wax, an active composition comprising a hydrogel, an active composition comprising an oleogel, an active composition comprising an organogel, or mixtures thereof. However, no use of liquid fragrance compositions is disclosed.

Consequently, a need exists for a simple and efficient fragrance delivery device that mitigates one or more of the aforementioned disadvantages of dispersing liquid fragrance compositions into the surrounding space through evaporation.

### SUMMARY OF THE INVENTION

In a first aspect, the present disclosure relates to a device comprising:
a) a body portion comprising a porous material,
   wherein the body portion has a volume and at least one surface,
   wherein the volume comprises at least one network of a plurality of fluidly connected passages,
   wherein the at least one network of fluidly connected passages has at least one first end and at least one second end,
   wherein the at least one first end and at least one second end are separated by a distance,
   wherein at least one of the first or second ends are fluidly connected to the at least one surface,
   wherein each individual passage within the plurality has a cross section,
   wherein the distance, and the cross section of each passage within the plurality defines a surface, and
b) at least one reservoir comprising a liquid fragrance composition fluidly connected to the body portion;
   wherein the fluid connection is configured to draw the liquid fragrance composition into the porous material of the body portion,
   wherein the porous material of the body portion is configured to absorb the liquid fragrance composition, and
   wherein the surface of the body portion is configured to disperse the liquid fragrance composition via evaporation.

In a second aspect, the present disclosure relates to a method of dispersing a liquid fragrance composition into a surrounding space via evaporation, comprising placing the device described herein into a space in need thereof, and allowing the liquid fragrance composition to evaporate from the device.

In a third aspect, the present disclosure relates to a kit, comprising:
a) a body portion comprising a porous material,
   wherein the body portion has a volume and at least one surface,
   wherein the volume comprises at least one network of a plurality of fluidly connected passages,
   wherein the at least one network of fluidly connected passages has at least one first end and at least one second end,
   wherein the at least one first end and at least one second end are separated by a distance,
   wherein at least one of the first or second ends are fluidly connected to the at least one surface,
   wherein each individual passage within the plurality has a cross section,
   wherein the distance, and the cross section of each passage within the plurality defines a surface, and
b) at least one reservoir comprising a liquid fragrance composition.

### BRIEF DESCRIPTION OF THE FIGURES

FIG. 1 shows an embodiment of the device according to some aspects of the present disclosure.

FIG. 2 shows another embodiment of the device according to some aspects of the present disclosure.

FIG. 3 shows yet another embodiment of the device according to some aspects of the present disclosure.

FIG. 4 shows the mass loss (in grams per day) of two comparative scaffolds.

FIG. 5 shows the mass loss (in grams per day) of two exemplary scaffolds according to the present disclosure over time.

FIG. 6 shows the mass loss (in grams per day) of another two exemplary scaffolds according to the present disclosure over time.

FIG. 7 shows the amount of mass loss (in grams per day) of an exemplary scaffold under various conditions.

FIG. 8 shows the amount of mass loss (in grams per day) of another exemplary scaffold under various conditions.

FIG. 9 shows the amount of mass loss (in grams per day) of yet another exemplary scaffold under various conditions.

FIG. 10 shows an exemplary device having a dual reservoir and double gyroid scaffold.

FIG. 11 shows two views of a fragrance delivery device featuring a central reservoir that can be refilled using any dispensing device.

FIG. 12 shows an exemplary fragrance delivery device having a single reservoir.

FIG. 13 shows another exemplary fragrance delivery device having a single reservoir.

FIG. 14 shows an exemplary fragrance delivery device made from ceramic materials.

FIG. 15 shows another exemplary fragrance delivery device made from ceramic materials.

FIG. 16 shows the average daily mass loss rate vs time of exemplary fragrance devices and various controls.

FIG. 17 shows three variations of a scaffold having a triply periodic minimal surface geometry with a connected wick.

FIG. 18 shows a variation of the scaffold having a triply periodic minimal surface geometry with a connected wick and a reservoir.

FIG. 19 shows the average daily mass loss rate vs time of exemplary fragrance devices with shell printed and normally printed scaffolds and connected wicks.

### DETAILED DESCRIPTION

The following detailed description sets forth various aspects and embodiments provided herein. The description is to be read from the perspective of the person of ordinary skill in the relevant art. Therefore, information that is well known to such ordinarily skilled artisans is not necessarily included. It would be apparent to ordinarily skilled artisans that the various aspects and embodiments provided herein may be combined in any manner without departing from the spirit of the disclosure.

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary. Unless defined otherwise, all technical and scientific terms used herein have the same meaning as is commonly understood by one of skill in the art to which this specification pertains.

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

As used herein, “or” is to be given its broadest reasonable interpretation and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

In the first aspect, the present disclosure relates to a device comprising:
a) a body portion comprising a porous material,
   wherein the body portion has a volume and at least one surface,
   wherein the volume comprises at least one network of a plurality of fluidly connected passages,
   wherein the at least one network of fluidly connected passages has at least one first end and at least one second end,
   wherein the at least one first end and at least one second end are separated by a distance,
   wherein at least one of the first or second ends are fluidly connected to the at least one surface,
   wherein each individual passage within the plurality has a cross section,
   wherein the distance, and the cross section of each passage within the plurality defines a surface, and
b) at least one reservoir comprising a liquid fragrance composition fluidly connected to the body portion;
   wherein the fluid connection is configured to draw the liquid fragrance composition into the porous material of the body portion,
   wherein the porous material of the body portion is configured to absorb the liquid fragrance composition, and
   wherein the surface of the body portion is configured to disperse the liquid fragrance composition via evaporation.

In some embodiments, at least one of the first or second ends are open. In some embodiments, each individual passage within the plurality has one or more branches.

The body portion of the device may have any cross-sectional shape, such as, for example, an irregular shape, a square shape, a rectangular shape, a circular shape, an elliptical shape, a rhomboid shape, a semi-circular shape, a trapezoidal shape and the like. Thus, in some embodiments, the body portion has a cross-sectional shape selected from the group consisting of: an irregular shape, a square, a rectangle, a circle, an ellipse, a rhombus, a semi-circle, and a trapezium.

Without intending to be limited to any particular theory, the surface provides a body portion having a structure having a porosity, a surface area and volume that may be configured to disperse the liquid fragrance composition.

As used herein, porosity includes macroporosity and microporosity. The terms “macroporosity”, “macroporous”, or “macropores” refer to pores having open pore sizes greater than or equal to 1 mm, typically greater than or equal to 5 mm, more typically greater than or equal to 10 mm. Without intending to be limited to any particular theory, this type of porosity allows air to penetrate deep into the center of the object, making it easier for fragrance compounds to evaporate. As will be described herein, macropores are defined by the surface geometry of the body portion of the device and unaffected by the method of manufacture.

On the other hand, the terms “microporosity”, “microporous”, or “micropores” relate to pores having open pore sizes less than 1 mm. In contrast to macropores, micropores arise from the manufacturing method used. For example, when powder bed fusion, such as sintering or multi jet fusion, is used in additive manufacturing, the selection of particle size of the powders used may result in a certain size of micropores.

As used herein, there are three different types of porosity: total porosity, closed porosity, and open porosity. Total porosity is the sum of open and closed porosity of a material. Closed porosity defines the ratio of the volume of the pores that are not connected to the outside air compared to the envelope volume. Herein, envelope volume represents the total volume of the object based on its outside dimensions (i.e., as if it was ‘shrink-wrapped’). Closed pores are not available for fluid transport or migration of the liquid fragrance oil or composition. Open porosity is a measure of the volume of open holes and pores that are connected to the outside air compared to the total envelope volume of an object. These pores are connected to the outside air and may enable fluid migration within the structure and through the surface.

The macropores are defined by the surface, which may be a triply periodic minimal surface geometry, or an analogue thereof. The surfaces represent the roots of equations that use periodic functions (e.g., sin, cos, tan) or hyperbolic functions (e.g. sinh, cosh, tanh) in three directions (x,y and z). These surfaces typically form many connected and undulating surfaces leading to an intertwined labyrinth. Interestingly, these also form an aesthetic and ‘organic’ look that is desirable. Examples of triply periodic minimal surface geometries suitable for use according to the present disclosure may be found, for example, in Gyroid and Gyroid-Like Surfaces: Rudolf, M., & Scherer, J. (2013), S. I. Publishing (Ed.), Double-Gyroid-Structured Functional Materials (pp. 7-19)). Additional examples of triply periodic minimal surface geometries suitable for use according to the aspects presented herein are disclosed in S. Andersson K. Larsson M. Larsson M. Jacob. (1999). Biomathematics, Mathematics of Biostructures and Biodynamics. Elsevier Science.

In some embodiments, the triply periodic minimal surface geometry is selected from the group consisting of: a gyroid geometry, a lidinoid geometry, a Schwarz D “diamond” geometry, or a Schwarz P “primitive” structure geometry.

In some embodiments, the surface is defined by a triply periodic minimal surface geometry is defined according to Equation 1:

$${φ}_{G} = F ( x , y , z ) = sin ( x ) \cdot cos ( y ) + sin ( y ) cos ( z ) + sin ( z ) cos ( x ) = T \qquad Equation 1$$

Varying the numerical value of T may vary the porosity, the surface area and/or volume of the body portion. For example, when T=0, the body portion is divided exactly into two separate enantiomeric interpenetrating single-gyroid volumes (both 50%). The two separate interpenetrating single-gyroid volumes each comprise a separate network of a plurality of hollow passages (referred to herein as “volume A” and “volume B”). When the value of T is between 0 and 1.413, the volume of volume A increases, while the volume of volume B decreases. Similarly, when the value of T is between 0 and −1.413 the opposite occurs, wherein the volume of volume B increases, while the volume of volume A decreases. When values of the absolute value of T is between 1.413 and 1.5, the surfaces are no longer connected. For absolute values of T that exceed 1.5, no realistic solution for Equation 1 exists.

In an embodiment, the value of T is selected from a numerical value between 0 and 1.43.

In another embodiment, the value of T is selected from a numerical value between 0 and −1.43.

In an embodiment, the at least one surface is defined by a triply periodic minimal surface geometry is defined according to Equation 2:

$$F ( x , y , z ) = ( {A}_{1} sin ( {B}_{1} x + {C}_{1} ) + {D}_{1} ) \cdot ( {A}_{2} cos ( {B}_{2} y + {C}_{2} ) + {D}_{2} ) + ( {A}_{3} sin ( {B}_{3} y + {C}_{3} ) + {D}_{3} ) \cdot ( {A}_{4} cos ( {B}_{4} z + {C}_{4} ) + {D}_{4} ) + ( {A}_{5} sin ( {B}_{5} z + {C}_{5} ) + {D}_{5} ) \cdot ( {A}_{6} cos ( {B}_{6} x + {C}_{6} ) + {D}_{6} ) = T , \qquad Equation 2$$

wherein A is amplitude; B is frequency, C is phase shift, D is vertical shift, and
wherein at least one of A, B, C, or D may vary in at least one of the x, y, or z direction of the body portion.

Using Equation 2 as an example, a basic sine wave of y=sin(x) can be modified as follows: y=A*sin(Bx+C)+D, where A-D represent parameters that alter the amplitude, the frequency, the phase shift and a vertical shift respectively. By altering these variables in one or all of the trigonometric functions of equation 2 (or similar equations), different geometries can be obtained.

In an embodiment, the at least one surface is defined by a triply periodic minimal surface geometry is defined according to Equation 3:

$${φ}_{D} = F ( x , y , z ) = sin ( x ) \cdot sin ( y ) \cdot sin ( z ) + sin ( x ) \cdot cos ( y ) \cdot cos ( z ) + cos ( x ) \cdot sin ( y ) \cdot cos ( z ) + cos ( x ) \cdot cos ( y ) \cdot sin ( z ) = 0 \qquad Equation 3$$

The surface may be defined by combining more than one equation that defines a triply periodic minimal surface geometry, as disclosed, for example, in Venkatesh, V., Reddy, K. A. K., & Sreekanth, E. (2014). Design of Mathematically Defined Heterogeneous Porous Scaffold Architecture for Tissue Engineering, 10 (24), 1169-1174. For example, the triply periodic minimal surface geometry may be defined by combining Equations 1 and 3 to arrive at Equation 4:

$${φ}_{mix} = µ \cdot {φ}_{G} + ( 1 - µ ) {φ}_{D} = 0 , \qquad Equation 4$$

wherein μ ranges from 0 to 1. In an embodiment, u is 0.5.

In an embodiment, the surface is defined by a triply periodic minimal surface geometry created by generative design and/or field-driven design. As used herein, generative design refers to the use of computational methods to generate triply periodic minimal surface geometries that satisfy desired parameters, such as performance or spatial requirements, materials, manufacturing methods, cost constraints, and the like. As used herein, field-driven design refers to the variation of a triply periodic minimal surface geometry according to one or more fields. Herein, a field is a distribution of points in 3D space, each point being assigned a value, and may be defined by a point (as in a field that varies radially), a plane, an implicit model, or simulation data, such as computational fluid dynamics data. The triply periodic minimal surface geometry is then varied spatially according to the one or more fields. For example, 3D computational fluid dynamics data, typically those simulating air currents, such as laminar air flow, turbulent air flow, convective air currents, and the like, may be used to generate an air velocity field, in which each point in the field represents an air velocity. The air velocity field is then used to create a triply periodic minimal surface geometry that has higher surface areas in areas of low air velocity and lower surface areas in high air velocity regions to provide a desired effect, such as more balanced and more uniform evaporation of a liquid fragrance composition. Suitable software for utilizing generative design and/or field-driven design include, but are not limited to, AutoCAD (Autodesk) or nTopology (nTopology, Inc.).

Triply periodic surfaces, such as the ones described herein, provide several advantages when used for dispensing liquid fragrance compositions. The triply periodic surfaces share a desirable trait of bifurcation, trifurcation, quadfurcation or even multifurcating (branching). Without wishing to be bound to any particular theory, it is contemplated that as a molecule evaporates from an internal surface there are numerous paths for this molecule to ‘find a way out’ of the labyrinth. As a molecule travels throughout the object, it has many ‘decision points’ to make that either steer it directly to the outside air or sometimes even deeper into the geometry. This randomization of path lengths can serve as a means of ‘mixing’ the fragrance further and should aid in linearizing the fragrance performance in terms of character and/or intensity. Such triply periodic surfaces have excellent mechanical strength and have been shown to have a relatively low pressure drop for flow throughout the object making it easy for air to move through the object despite its high surface area. Another advantage of such geometries is prevention of plugging or blocking. There are so many paths for the liquid to travel, that a block/restriction in one channel allows continued flow in many other pathways.

The cross section of each individual passage within the plurality varies in at least one of the x, y, or z direction of the body portion. In an embodiment, the cross section of each individual passage within the plurality is greater in the center of the body portion than the cross section of each individual passage within the plurality at the periphery of the body portion.

In another embodiment, the cross section of each individual passage within the plurality is greater at the periphery of the body portion than the cross section of each individual passage within the plurality at the center of the body portion. Without intending to be limited to any particular theory, the variation of the cross section of each individual passage within the plurality may alter the evaporation rate of the liquid fragrance composition.

Without intending to be limited to any particular theory, the cross section of each individual passage within the plurality may be altered in at least one of the x, y, or z direction of the body portion, thereby generating a body portion having a radial porosity gradient. In this instance, the term “porosity gradient” refers to the variation in the cross section of each individual passage within the plurality in at least one of the x, y, or z direction of the body portion.

In an embodiment, the cross section of each individual passage within the plurality may be altered by varying the frequency parameter in any one of Equations 1 to 4 in at least one of the x, y, or z direction of the body portion.

In an embodiment, the cross section of each individual passage within the plurality may be altered by varying the amplitude parameter in any one of Equations 1 to 4 in at least one of the x, y, or z direction of the body portion.

In an embodiment, the cross section of each individual passage within the plurality may be altered by varying the phase shift parameter in any one of Equations 1 to 4 in at least one of the x, y, or z direction of the body portion.

In an embodiment, the cross section of each individual passage within the plurality may be altered by varying the vertical shift parameter in any one of Equations 1 to 4 in at least one of the x, y, or z direction of the body portion.

In an embodiment, the cross section of each individual passage within the plurality may be altered by varying u in Equation 4 above as a function of distance in at least one of the x, y, or z direction of the body portion, wherein μ ranges from 0 to 1.

In an embodiment, the cross section of each individual passage within the plurality may be altered by varying u in Equation 4 and introducing porosity gradients in at least one of the x, y, or z direction of the body portion.

In some embodiments, the cross section of each individual passage within the plurality is at least 1 mm, typically at least 5 mm, more typically at least 10 mm.

In an embodiment, the body portion comprises two networks of a plurality of fluidly connected passages. In an embodiment, the first and second networks do not interconnect.

In some embodiments, the body portion comprises three networks of a plurality of fluidly connected passages. In an embodiment, the first, second, and third networks do not interconnect.

The device may be configured to be compact with a small footprint while having a high surface area. Thus, in some embodiments, the device has a surface area to volume ratio of at least 1 cm²:cm³, or at least 2 cm²:cm³, or at least 3 cm²:cm³, or at least 4 cm²:cm³, or at least 5 cm²:cm³. In some embodiments, the device has a surface area to volume ratio of at least 6 cm²:cm³, or at least 7 cm²:cm³, or at least 8 cm²:cm³, or at least 9 cm²:cm³, or at least 10 cm²:cm³.

In some embodiments, random straight lines drawn through the center of a device intersect on average at least 2, or 3, or 4, or more times with the at least one surface. In some embodiments, random straight lines drawn through the center of a device intersect on average at least 5, or 10, or 20, or more times with the at least one surface.

As described herein, open porosity is a measure of the volume of open holes and pores that are connected to the outside air compared to the total envelope volume of an object. These pores are connected to the outside air and may enable fluid migration within the structure and through the surface. Open pore porosity may be measured according to any methods known to those of ordinary skill in the art. For example, a helium pycnometer may be used in which helium gas penetrates into the open pores of the material on application of pressure. Therefore, it is able to ‘see’ the closed pore porosity and volume of the material itself. By comparing this to the envelope volume, the open pore porosity can be determined. Another exemplary method may be the use of mercury porosimetry, which is based on the intrusion of mercury into a porous structure under controlled pressure to measure the open pore volume as well as pore size and size distribution.

In some embodiments, the body portion has an open pore porosity of 0.01 to 0.9.

In some embodiments, the body portion comprises a plurality of pores, the pores having a size of less than 1,000 μm.

The body portion comprises a porous material, typically microporous material. Suitable porous materials for the body portion include, but are not limited to, porous porcelain materials, plastics, molded ceramics, glass fibers, clay, activated carbon, cellulose, wood materials, such as wood pulp and wood fiber, and any combination thereof.

Plastics suitable for use according to the present disclosure may be thermoplastic and/or thermoset materials. As understood by the ordinarily-skilled artisan, thermoplastic materials are materials that become pliable or moldable at a certain elevated temperature and solidifies upon cooling and thermoset materials are materials obtained by irreversibly hardening (“curing”) a soft solid or viscous liquid prepolymer. Plastics suitable for use according to the present disclosure include, but are not limited to, acrylonitrile styrene acrylate (ASA), acrylonitrile butadiene styrene (ABS), polystyrenes, polylactic acid (PLA), polycarbonates, polyether sulfones, polyvinyl chloride (PVC), polyvinylidene fluoride (PVDF), polytetrafluoroethylenes, polyphenylene sulfides, polyamides, such as polyphthalamides and nylon; polyesters, such as polyethylene terephthalate; polypropylenes, polyacrylates, polysulfones, polyurethanes, polyetherimides, polyesterimides, and polyaryl ether ketones, such as polyether ether ketones and polyether ketone ketones; and any combination or copolymer thereof.

In an embodiment, the body portion comprises nylon, polypropylene, or a combination thereof.

The porous material may further comprise support materials that are removed during post-processing and include, but are not limited to water-dissolvable materials, such as polyvinyl alcohol (PVA); breakaway materials, and wax.

The porous material may further comprise a biodegradable material, such as bioplastics. Exemplary bioplastics include, but are not limited to, starch-based plastics, cellulose-based plastics, protein-based plastics, aliphatic polyesters, such as polylactic acid, polyamide 11, bio-derived polyethylene, and the like.

The porous material may other suitable materials selected from the materials disclosed in Wohler, T. (2016). Wohlers Report 2016 3D Printing and Additive Manufacturing State of the Industry. Annual Worldwide Progress Report. Wohlers Associates, Inc.).

In an embodiment, the porous material is selected from the group consisting of, plastics, metals, uv-cured polymers, and mixtures thereof.

The body portion may be formed by any suitable method, readily selected by one of ordinary skill in the art. Non-limiting examples of methods to form the body portion include casting, additive manufacturing (“3D printing”), injection molding and the like.

As would be understood by a person of ordinary skill in the art, objects manufactured by additive manufacturing methods are typically created in 3D modeling or CAD software, for example ShapeJS (Shapeways), Blender (Blender Foundation), AutoCAD (Autodesk), Solidworks (Dassault Systèmes), nTopology (nTopology, Inc.), and the like. The resulting geometry is then manufactured accordingly.

Exemplary additive manufacturing methods suitable for use according to the present disclosure include, but are not limited to, extrusion, such as fused deposition modeling; resin curing processes, such as stereolithography with direct laser writing, digital light processing (DLP), continuous liquid interface production (CLIP), and continuous digital light manufacturing; powder bed fusion, such as selective laser sintering, binder jetting, material jetting and multi jet fusion; and the like.

In an embodiment, the body portion and/or wick are each formed by powder bed fusion, typically selective laser sintering or multi jet fusion. In an embodiment, the body portion and/or wick are each formed by shell printing. As used herein, “shell printing” (also called “skin-coring”) refers to a powder bed fusion technique in which only a thin skin-layer is 3D printed, resulting in the enclosure of unfused powder on the inside of the object. Without wishing to be bound by theory, shell printing allows for increased open pore porosity. When shell printing is used, the unfused powder on the inside should not be inaccessible to the liquid fragrance composition. Accordingly, in some embodiments, the body portion and/or wick each include one or more entry holes that are big enough for liquid to enter, but small enough for powders to stay inside the geometry. In an embodiment, the entry holes are less than 1 mm in diameter, typically between 0.01 to 1 mm, more typically 0.1 to 1 mm.

The structure of the body portion may be defined by at least one solid surface. In some embodiments, the structure of the body portion may be defined by at least one perforated surface. Examples of perforated surfaces include, but are not limited to wireframes, tessellated shapes, fibers, trabecular structures, and the like.

The device of the present disclosure comprises at least one reservoir comprising a liquid fragrance composition fluidly connected to the body portion.

As used herein, the reservoir may be any space or void capable of holding a liquid fragrance composition. The space or void acting as a reservoir may be provided by a container or may be a space or void inside the body portion of the device. A container acting as reservoir may be contructed from a material impermeable to liquids, such as, for example, glass or plastic, and typically comprises an opening through which the liquid fragrance composition is fluidly connected to the body portion. In an embodiment, the body portion is external to the reservoir and the liquid fragrance composition is drawn from the reservoir to the body portion of the device through a fluid connection.

In another embodiment, the body portion is partially immersed in the liquid fragrance composition inside the reservoir. In such an embodiment, the body portion is in direct contact with the liquid fragrance composition, which is drawn into the body portion from which the liquid fragrance composition is dispersed via evaporation.

In some embodiments, the reservoir is a space or void inside the body portion of the device. In such an embodiment, the body portion is in direct contact with the liquid fragrance composition, which is drawn into the body portion from which the liquid fragrance composition is dispersed via evaporation. It is envisioned, in some embodiments, that the internal space or void is surrounded by a region of low porosity that is fluidly connected to a region of high porosity. The region of low porosity acts as a ‘well’ but is at least partially permeable by the liquid fragrance composition. In some embodiments, a coating may be disposed onto the bottom of the body portion of the device, thereby preventing leaking.

In some embodiments, the reservoir is provided by a liquid-permeable container, such as a container made from a porous material, inside the body portion of the device. In such an embodiment, the body portion is in fluid contact with the liquid fragrance composition through the walls of the liquid-permeable container inside the body portion. The liquid fragrance composition is drawn into the body portion, from which the liquid fragrance composition is dispersed via evaporation. In some embodiments, a coating may be disposed onto the bottom of the body portion of the device, thereby preventing leaking.

In some embodiments, the device according to the present disclosure comprises two or more reservoirs, typically two reservoirs. In such an embodiment, the device comprises two or more liquid fragrance compositions, each reservoir having one liquid fragrance composition. In an embodiment, the two or more liquid fragrance compositions are the same. In another embodiment, the two or more liquid fragrance compositions are different.

As used herein, the term “liquid fragrance composition” refers to a liquid which is at least partially volatile, i.e., can evaporate, and which is able to impart a fragrance or other benefit to the surrounding space.

The liquid fragrance composition is a low viscosity liquid at room temperature (25° C.). Thus, in an embodiment, the liquid fragrance composition has a viscosity of about 0.1 to about 10,000 mPa·s. In an embodiment, the liquid fragrance composition has a viscosity of about 0.1 to about 1,000 mPa·s. In an embodiment, the liquid fragrance composition has a viscosity of about 0.1 to about 100 mPa·s.

The liquid fragrance composition should have sufficient wetting between the fragrance liquid and the porous material that used in the body portion of the device. In some instances, the porous materials used in the device may be surface treated or selected in a way to improve the wetting angle that would allow faster or more complete wetting. A person having ordinary skill in the art would be able to use a sessile drop tensiometer to determine the wetting angle between the fragrance liquid and the porous material and make formulation changes accordingly, as desired.

In an embodiment, the liquid fragrance composition has a density of from about 0.7 to about 1.3 g/mL. In an embodiment, the liquid fragrance composition has a surface tension of about 10 to about 70 mN/m.

The liquid fragrance composition may contain between 40% by weight and 100% by weight fragrance, which typically comprise chemicals or essential oils. In an embodiment, the liquid fragrance composition comprises from 60% by weight to 100% by weight of fragrance. The balance of these formulations can include solvents, dyes, colorants, antioxidants, UV inhibitors, bittering agents, etc. as are generally known to those of ordinary skill in the art.

In some embodiments, the liquid fragrance composition is a perfume. As perfume there can be used any ingredient or mixture of ingredients currently used in perfumery, i.e., capable of exercising a perfuming action. More often, however, a perfume will be a more or less complex mixture of ingredients of natural or synthetic origin. The nature and type of the ingredients do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitrites, terpene hydrocarbons, nitrogenous or sulphurous heterocyclic compounds and essential oils of natural or synthetic origin. Many of these ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery.

In some embodiments, the perfuming action may further comprise providing a sensorial and/or emotional benefit, or, alternatively, the perfuming action may be configured to prevent the habituation of the user to the perfume. The sensorial and/or emotional benefit may be provided by the addition of an additional agent to the liquid fragrance composition. For example, by way of illustration, the fragrance composition may further comprise a cooling compound, that imparts a cooling sensation to a user.

Although special mention has been made hereinabove of the perfuming effect that can be exerted by the devices of the present disclosure, the same principles apply to analogous devices for the diffusion of deodorizing or sanitizing vapors, the perfume being replaced by a deodorizing composition, an antibacterial, an insecticide, an insect repellent or an insect attractant. As used herein, the term “sanitizing vapors”, refers to the vapors of those substances which can enhance the degree of acceptance of the air surrounding the observer, but also to those substances which can exert an attractant or repellent effect towards certain species of insects, for instance towards houseflies or mosquitoes, or else, which can have bactericide or bacteriostatic activity. Mixtures of such agents can also be used.

The liquid fragrance composition may also contain optional ingredients acting as, for example, solvents, thickeners, antioxidants, dyes, bittering agents and UV inhibitors.

In some embodiments, the liquid fragrance composition further comprises one or more solvents. The one or more solvents may be useful to provide a single-phase liquid and/or to modulate the speed of evaporation of the liquid fragrance composition into the surrounding air. The solvents may belong to the families of isoparaffins, paraffins, hydrocarbons, glycols, glycol ethers, glycol ether esters, esters or ketones.

Examples of commercially available solvents suitable for use in the present disclosure include the solvents known under the tradename Isopar® H, J, K, L, M, P or V (isoparaffins; origin: Exxon Chemical), Norpar® 12 or 15 (paraffins; origin: Exxon Chemical), Exxsol® D 155/170, D 40, D 180/200, D 220/230, D 60, D 70, D 80, D 100, D 110 or D 120 (dearomatised Hydrocarbons; origin: Exxon Chemical), Dowanol® PM, DPM, TPM, PnB, DPnB, TPnB, PnP or DPnP (glycol ethers; origin: Dow Chemical Company), Eastman® EP, EB, EEH, DM, DE, DP or DB (glycol ethers; origin: Eastman Chemical Company), Dowanol® PMA or PGDA (glycol ether esters; origin: Dow Chemical Company) or Eastman® EB acetate, Eastman® DE acetate, Eastman® DB acetate, Eastman EEP (all glycol ether esters; all origin: Eastman Chemical Company).

Other solvents suitable for use in the present disclosure include dipropylene glycol, propylene glycol, ethylene glycol ethyl ether acetate, ethylene glycol diacetate, isopropyl myristate, diethyl phthalate, 2-ethylhexyl acetate, methyl n-amyl ketone or di-isobutyl ketone.

The total amount of solvents present in the liquid fragrance composition may vary between 0.0% and 80%, alternatively between 30% and 70%, by weight relative to the total weight of the liquid fragrance composition.

The liquid fragrance composition may optionally comprise a thickener so long as the viscosity of the liquid fragrance composition is not so high that the composition is prevented from being drawn into the body portion or results in clogging. Non-limiting examples of useful thickener ingredients include ethyl cellulose (commercial examples of which are available from Hercules Inc.), fumed silica (commercial examples of which are available from Degussa) and styrene-butadiene-styrene block copolymers (commercial examples of which are available from Shell).

In some embodiments, the total amount of thickeners present in the liquid fragrance composition may vary between 0.0% and 10%, alternatively between 1% and 4%, by weight relative to the total weight of the liquid fragrance composition.

Non-limiting examples of useful antioxidant ingredients include sterically hindered amines, i.e., the derivatives of the 2,2,6,6-tetramethyl-piperidine, such as those known under the tradename Uvinul® (origin BASF AG) or Tinuvin® (origin: Ciba Speciality Chemicals), as well as the alkylated hydroxyarene derivatives, such as butylated hydroxytoluene (BHT).

In some embodiments, the total amount of antioxidants present in the liquid fragrance composition may vary between 0.0% and 10%, alternatively between 1% and 4%, by weight relative to the total weight of the liquid fragrance composition.

The liquid fragrance composition may comprise other optional ingredients, such as dyes. Suitable dyes may be oil-soluble and can be found in the Colour Index International, published by The Society of Dyers and Colourist. Non-limiting examples of suitable dyes include derivatives of the anthraquinone, methine, azo, triarylmethane, triphenylmethane, azine, aminoketone, spirooxazine, thioxanthene, phthalocyanine, perylene, benzopyran or perinone families. Examples of such dyes which are commercially available are known under the tradename Sandoplast® Violet RSB, Violet FBL, Green GSB, Blue 2B or Savinyl® Blue RS (all anthraquinone derivatives; origin: Clariant Huningue S.A.), Oilsol® Blue DB (anthraquinone; origin: Morton International Ltd.), Sandoplast® Yellow 3G (methine; origin: Clariant Huningue S.A.), Savinyl® Scarlet RLS (azo metal complex; origin: Clariant Huningue S.A.), Oilsol® Yellow SEG (monoazo; origin: Morton International Ltd.), Fat Orange® R (monoazo; origin: Hoechst AG), Fat Red® SB (diazo; origin: Hoechst AG), Neozapon® Blue 807 (phtalocyanine; origin: BASF AG), Fluorol® Green Golden (perylene; origin: BASF AG).

In some embodiments, the total amount of dyes present in the liquid fragrance composition may vary between 0.0% and 0.5%, alternatively between 0.005% and 0.05%, by weight relative to the total weight of the liquid fragrance composition.

A bittering agent may be desirable to render the product unpalatable, making less likely that the liquid fragrance composition is ingested, especially by young children. Non-limiting examples of bittering agents include isopropyl alcohol, methyl ethyl ketone, methyl n-butyl ketone or yet a denatonium salt such as the denatonium benzoate known also under the trademark Bitrex™ (origin: Mac Farlan Smith Ltd.).

The bittering agent may be incorporated in the liquid fragrance composition in an amount of from 0.0% to 5%, by weight relative to the total weight of the liquid fragrance composition. In the case of Bitrex™, the bittering agent may be incorporated in the liquid fragrance composition in an amount of from 0.0% and 0.1%, alternatively between 0.001% to 0.05%, by weight relative to the total weight of the liquid fragrance composition.

Non-limiting examples of useful UV-inhibitor ingredients, include benzophenones, diphenylacrylates or cinnamates such as those available under the trade name Uvinul® (origin: BASF AG).

In some embodiments, the total amount of UV-inhibitors present in the active composition may vary between 0.0% and 0.5%, alternatively between 0.01% and 0.4%, by weight relative to the total weight of the liquid fragrance composition.

The device according to the present disclosure may further comprise a wick. In some embodiments, the at least one reservoir comprising the liquid fragrance composition is fluidly connected to the body portion through at least one wick.

The at least one wick is made from any material known to those of ordinary skill in the art that is capable of passively moving a liquid against gravity. The migration of liquid, such as the liquid fragrance composition, may occur through capillary action, wicking action, and/or absorption. In an embodiment, the at least one wick comprises a porous material, typically microporous material.

Suitable porous materials used for constructing the at least one wick include, but are not limited to, paper, porous porcelain materials, plastics, plastic fibers, plastic foams, molded ceramics, glass fibers, clay, activated carbon, cellulose, wood materials, such as wood pulp and wood fibers, and any combination thereof.

Plastics, plastic fibers, and plastic foams suitable for use in the wick include, but are not limited to, acrylonitrile styrene acrylate (ASA), acrylonitrile butadiene styrene (ABS), polystyrenes, polylactic acid (PLA), polycarbonates, polyether sulfones, polyvinyl chloride (PVC), polyvinylidene fluoride (PVDF), polytetrafluoroethylenes, polyphenylene sulfides, polyamides, such as polyphthalamides and nylon; polyesters, such as polyethylene terephthalate; polypropylenes, polyacrylates, polysulfones, polyurethanes, polyetherimides, polyesterimides, and polyaryl ether ketones, such as polyether ether ketones and polyether ketone ketones; and any combination or copolymers thereof.

It is envisioned that the liquid fragrance composition and the material used in the device are compatible such that the fragrance does not dissolve in or swell the material, typically plastic material. If the plastic swells it may cause undesirable blockage. Thus, in some embodiments, the fragrance is such that it does not dissolve in or swell the plastic material.

Hansen solubility parameters, for example, may be used advantageously to predict compatibility between fragrance and plastic, and to formulate fragrance compositions to ensure good compatibility.

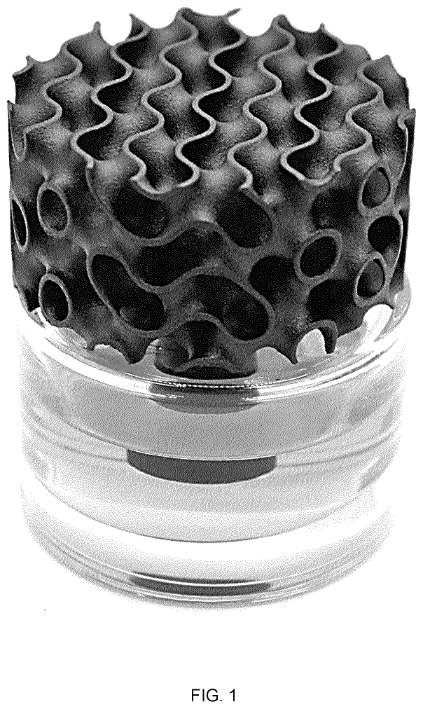

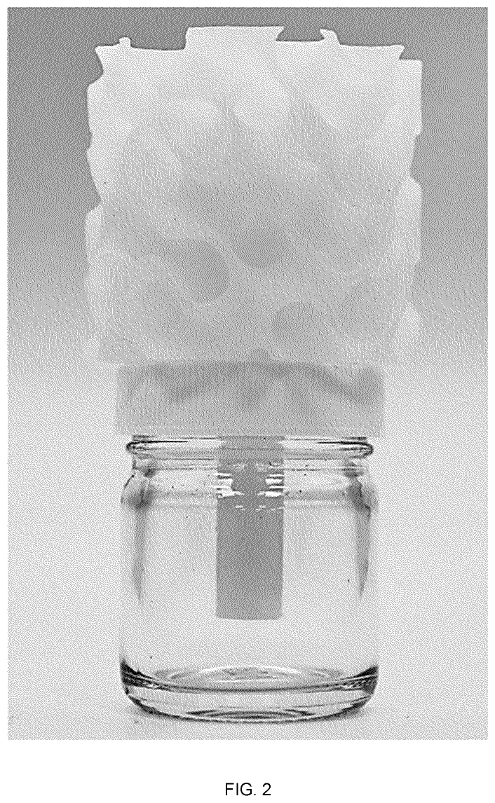

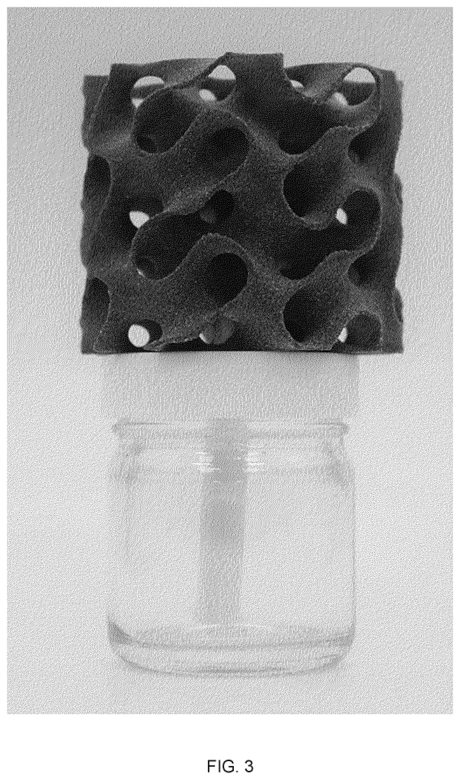

The wick may be an externally added wick or it may be integrated, i.e., built-in, as part of the body portion during production. In the case when the wick is integrated as part of the body portion during production, it may be fixed or it may be manufactured with a hinge or other mechanical design that allows the wick to be moved into the reservoir, and consequently into the liquid fragrance composition, without assembly. FIGS. 1 and 2 show exemplary devices in which the wick is integrated as part of the body portion during production. FIG. 3 shows an exemplary device in which the wick is externally added.

The porous material, typically microporous material, used for the wick may be the same as or different from the porous material used in the body portion. In some embodiments, the porous material, typically microporous material, used for the wick is the same as the porous material used in the body portion.

The wick may have any shape suitable for use in the device of the present disclosure. The wick may have a cross sectional area that is constant, such as a cylindrical shape, or one that varies with the liquid level in the respective reservoir.

The wick may be sized in such a manner that it is not a bottleneck for total evaporation using methods known to those of ordinary skill in the art. For example, the maximum flow rate through a wick can be modelled by equations that are a function of the cross-sectional area, A, of a wick following Equation 5:

$${Q}_{max} = A \frac{K}{μ} ( \frac{3 ( 1 - ε ) γ cos θ}{ε {HR}_{b}} - ρ g ) \qquad Equation 5$$

wherein Qₘₐₓ represents the maximum flow rate, A is the cross-sectional area, K is the permeability coefficient, μ is the liquid dynamic viscosity, ε is the porosity, γ is the liquid surface tension and θ represents the contact angle, H is the wicked liquid height, R_{b} represents the mean bead radius, ρ is the liquid density and g is gravitational acceleration.

Therefore, a non-cylindrical wick with a restriction in cross sectional area may be desirable, especially if the wick could be lowered/or raised slightly exposing a higher or lower surface area at the liquid meniscus (see Beyhaghi, S., Geoffroy, S., Prat, M. and Pillai, K. M. (2014), Wicking and evaporation of liquids in porous wicks: A simple analytical approach to optimization of wick design. AICHE J., 60:1930-1940. https://doi.org/10.1002/aic.14353).

In some embodiments, the device according to the present disclosure comprises two or more reservoirs, typically two reservoirs. In such an embodiment, the device comprises two or more liquid fragrance compositions, each reservoir having one liquid fragrance composition. Accordingly, in some embodiments, the device may comprise two or more wicks fluidly connecting the liquid fragrance composition in each reservoir and the body portion.

The triply periodic surfaces of the device described herein provide, in some embodiments, for a double or triple (or more) gyroid structure which allows for a compact device with multiple wicks connected to separate intertwined emanating surfaces all releasing fragrances independently (in rate and character) without the liquids ever interacting with one another. It is even conceivable that if one or both fragrances have different colorants added that interesting visual effects can be obtained when the colorant is wicked into the porous body portion and provides an aesthetic color contrast effect.

For example, by way of illustration, a first liquid fragrance composition may have a first olfactive note, and a second liquid fragrance composition may have a second olfactive note, different from the first olfactive note. In some embodiments, the device may be configured to release the first liquid fragrance composition at a different rate than the second liquid fragrance composition. In some embodiments, the device may be configured to release the first liquid fragrance composition and the second liquid fragrance composition at a rate that maintains the perception of a particular olfactive note at a consistent level over time.

In another illustrative example, the device may comprise a liquid fragrance composition comprising a fragrance ingredient in reservoir A and a liquid fragrance composition comprising a malodor counteracting agent in reservoir B. The two components may be chemically incompatible with one another but may be released simultaneously by the device without ever coming into contact with one another. The malodor counteractant may have a light blue color to be congruent with ‘cleaning’ whereas the fragrance may be colored purple to communicate a ‘flowery’ effect to support the flower fragrance.

Without intending to be limited to any particular theory, the device may be configured to release the first liquid fragrance composition at a different rate than the second liquid fragrance composition by providing a structure having an increased surface area in volume A, compared to volume B. Alternatively, the device may be configured to release the first liquid fragrance composition at a different rate than the second liquid fragrance composition by providing a structure having a decreased pore size in volume A, compared to volume B. Alternatively, the device may be configured to release the first liquid fragrance composition at a different rate than the second liquid fragrance composition by providing a structure having an increased surface area to volume ratio in volume A, compared to volume B.

While the device of the present disclosure can readily dispense the liquid fragrance composition in a passive manner, the rate of evaporation may be increased using external devices. In an embodiment, the device further comprises an apparatus for increasing evaporation, typically a heating element, a fan, a pump, a rotating stand, an oscillating stand, or a translating stand.

In the second aspect, the present disclosure relates to a method of dispensing a liquid fragrance composition into a surrounding space, comprising placing the device described herein into a space in need thereof, and allowing the liquid fragrance composition to evaporate from the device.

Placing the device described herein into a space in need thereof, and allowing the liquid fragrance composition to evaporate from the device may be achieved according to any methods known to those of ordinary skill in the art.

The rate of evaporation may be increased using external devices. In an embodiment, the method further comprises increasing evaporation using an apparatus, typically a heating element, a fan, a pump, a rotating stand, an oscillating stand, or a translating stand.

The device is particularly suited to allow for natural convection to occur within the macropores. This can be further accelerated by differential heating of the device, for example, by placing it in the sun on a windowsill. As the sun heats up the surfaces of the body portion of the device at different rates, natural convection will induce air movements within the device allowing for faster evaporation to occur.

The features of the device described herein are applied mutatis mutandis to the method.

In the third aspect, the present disclosure relates to a kit, comprising:
a) a body portion comprising a porous material,
   wherein the body portion has a volume and at least one surface,
   wherein the volume comprises at least one network of a plurality of fluidly connected passages,
   wherein the at least one network of fluidly connected passages has at least one first end and at least one second end,
   wherein the at least one first end and at least one second end are separated by a distance,
   wherein at least one of the first or second ends are fluidly connected to the at least one surface,
   wherein each individual passage within the plurality has a cross section,
   wherein the distance, and the cross section of each passage within the plurality defines a surface, and
b) at least one reservoir comprising a liquid fragrance composition.

In an embodiment, the kit further comprises at least one wick configured to fluidly connect the at least one reservoir with the body portion.

In some embodiments, the reservoir holds and protects the liquid fragrance from evaporation, spilling and oxidation/reactions prior to use. Generally, the reservoir will be closed before use (i.e., provided with a tamper-evident seal and cap). When the device is activated by a consumer by removing the seal and cap and inserting the wick into the liquid fragrance composition, fluidly connecting the reservoir and liquid fragrance composition with the body portion, the reservoir merely holds the liquid, preventing too rapid evaporation and spillage.

The features of the device described herein are applied mutatis mutandis to the kit just described.

The devices, kits, methods, and processes according to the present disclosure are further illustrated by the following non-limiting examples.

### Example 1. Mass Loss of Devices without Reservoir

Scaffolds were designed using a gyroid geometry defined by the implicit function:

$$sin ( x ) * cos ( y ) + sin ( y ) * cos ( z ) + sin ( z ) * cos ( x ) = 0$$

and produced by 3D printing using powder bed fusion.

Two different scaffolds were evaluated-one made of polypropylene (Scaffold A) and one made of nylon (Scaffold B). Fragrance oil was added to both the scaffolds using a pipette to saturate the scaffolds. All the materials involved were pre-weighed to accurately measure the fragrance oil mass loss at the end. The measured mass before and after addition of fragrance oil are summarized in Table 1 below.

TABLE 1

| Sample | Mass, g |
|---|---|
| Scaffold A (Polypropylene), | 12.42 |
| unsaturated | |
| Scaffold A saturated with oil | 13.04 |
| Scaffold B (Nylon), unsaturated | 12.48 |
| Scaffold B saturated with oil | 13.50 |

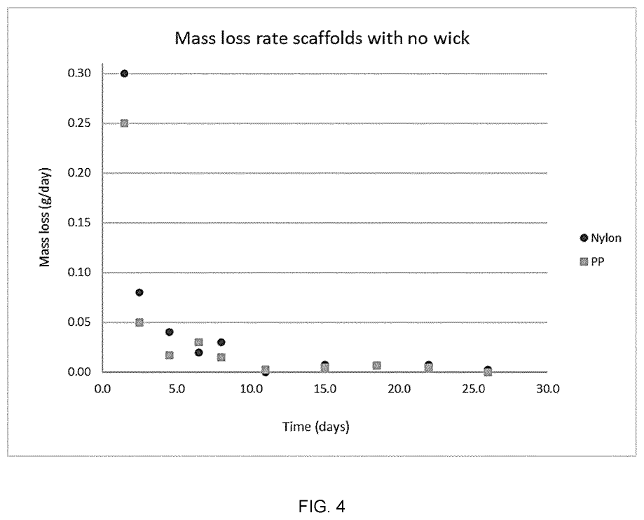

The saturated scaffolds were allowed to disperse the fragrance composition through evaporation. The masses of the saturated scaffolds were measured over a period of up to 30 days. FIG. 4 shows the mass loss (in grams per day) of scaffold A and scaffold B. Both scaffolds were quickly saturated with the oil. As shown in FIG. 4, the rate of weight loss shown in both structures peaked on day 1, then gradually decreased and stabilized by day 4 as the fragrance oil logically depleted. This example shows that the scaffolds are capable of releasing fragrance at a good rate, but good fragrance release is not sustained.

### Example 2. Mass Loss of Exemplary Devices with Printed Wick and Reservoir

The scaffolds of the present example we produced according to the procedure described in Example 1, except that the gyroid scaffold is joined with a cylinder, which acts as an integrated wick.

Two exemplary scaffolds—one made up of nylon (Scaffold C) and one made up of polypropylene (Scaffold D)—were produced, each having an integrated 3D printed wick and each having a reservoir. The reservoir was charged with fragrance oil and the wick portion of the respective scaffold was inserted into the reservoir. All the materials involved were pre-weighed to accurately measure the fragrance oil mass loss at the end of the evaluation. The measured mass of all the materials for each exemplary device are summarized in Tables 2 and 3 below.

TABLE 2

| Sample | Mass, g |
|---|---|
| Scaffold C (Nylon) | 24.66 |
| with printed wick | |
| Glass jar without lid | 51.12 |
| Lid | 3.09 |
| Fragrance oil | 23.00 |

TABLE 3

| Sample | Mass, g |
|---|---|
| Scaffold D (polypropylene) | 21.88 |
| with printed wick | |
| Glass jar without lid | 52.46 |
| Lid | 3.04 |
| Fragrance oil | 23.01 |

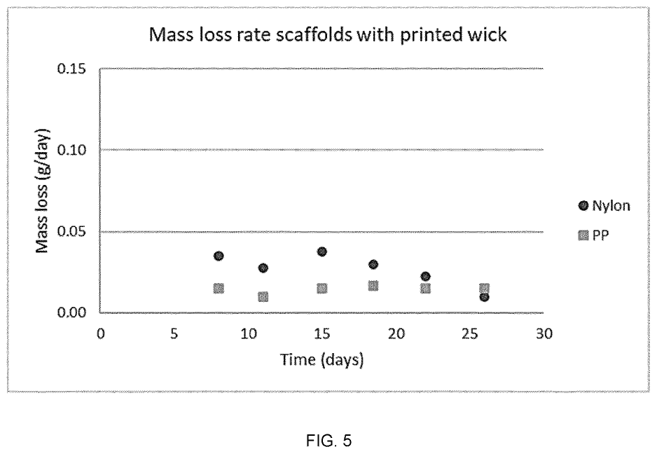

The scaffolds were allowed to sit for a few days to allow sufficient transfer of fragrance oil to the surfaces. The saturated scaffolds were allowed to disperse by evaporation the fragrance composition and the masses of the saturated scaffolds were measured over a period of up to 30 days. FIG. 5 shows the mass loss (in grams per day) of scaffold C and scaffold D over time. Data up to day 7 were not used as the scaffolds did not reach saturation until day 7. As shown in FIG. 5, both scaffolds showed a generally constant rate of fragrance loss.

### Example 3. Mass Loss of Exemplary Devices with External Wick and Reservoir

Two exemplary scaffolds—one made up of nylon (Scaffold E) and one made up of polypropylene (Scaffold F)—were produced, each having an external wick and each having a reservoir. The external wick used was made of paper wrapped polyester fiber and is representative of a commercially available wick used in air fresheners. The reservoir was charged with fragrance oil and the wick portion of the respective scaffold was inserted into the reservoir. All the materials involved were pre-weighed to accurately measure the fragrance oil mass loss at the end of the evaluation. The measured mass of all the materials for each exemplary device are summarized in Tables 4 and 5 below.

TABLE 4

| Sample | Mass, g |
|---|---|
| Scaffold E (nylon) | 13.08 |
| Wick | 0.83 |
| Glass jar without lid | 50.74 |
| Lid | 3.1 |
| Fragrance oil | 23.02 |

TABLE 5

| Sample | Mass, g |
|---|---|
| Scaffold F (polypropylene) | 15.33 |
| Wick | 0.83 |
| Glass jar without lid | 52.29 |
| Lid | 3.15 |
| Fragrance oil | 23.00 |

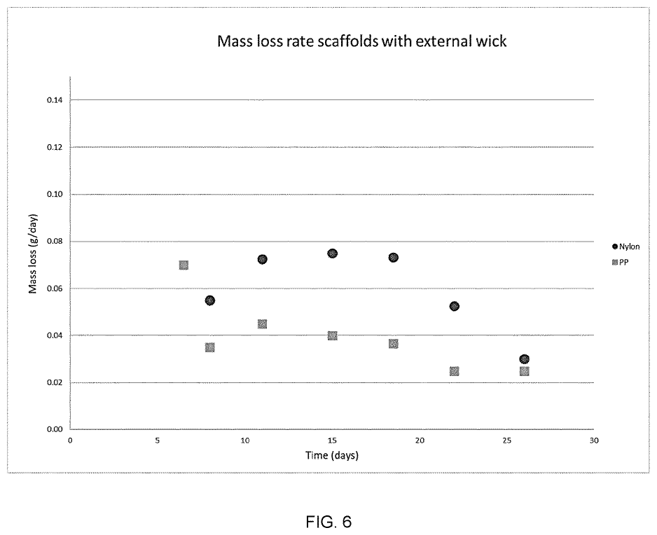

The scaffolds were allowed to sit for a few days to allow sufficient transfer of fragrance oil to the surfaces. The saturated scaffolds were allowed to evaporatively disperse the fragrance composition and the masses of the saturated scaffolds were measured over a period of up to 30 days. FIG. 6 shows the mass loss (in grams per day) of scaffold E and scaffold F over time. Data up to day 7 were not used as the scaffolds did not reach saturation until day 7.

### Example 4. Mass Loss of Exemplary Device with Printed Wick and Reservoir Under Various Conditions

Scaffold C according to Example 2 (nylon scaffold with printed wick) was subjected to various conditions: stationary state, positioning on a rotating disc, and positioning under a fan to evaluate fragrance loss. Scaffold C was placed at a fixed position on a rotating disc platform and mass loss amount was noted while alternating on a regular basis over several days the following conditions: stationary state, rotating disc state and fan state. The scaffold was pre-weighed to accurately determine the fragrance oil mass loss results at the end. Fan speed was set to 1700 RPM (60 CFM) and rotating disc speed was 7.5 rpm, with the edge of the device placed at the edge of the disc (22 cm from the center).

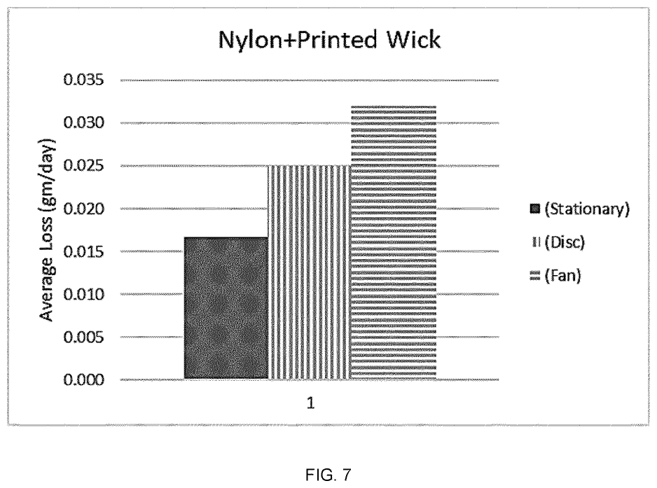

FIG. 7 shows the amount of mass loss (in grams per day) under varying conditions. As shown in FIG. 7, the mass loss varies based on the condition scaffolds were exposed to. The stationary state showed the lowest fragrance loss, followed by rotating disc and fan giving the highest fragrance loss.

### Example 5. Mass Loss of Exemplary Device with External Wick and Reservoir Under Various Conditions

Scaffold E according to Example 3 (nylon scaffold with external wick) was subjected to various conditions: stationary state, positioning on a rotating disc, and positioning under a fan to evaluate fragrance loss. Scaffold E was placed at a fixed position on a rotating disc platform and mass loss amount was noted while alternating on a regular basis over several days the following conditions: stationary state, rotating disc state and fan state. The scaffold was pre-weighed to accurately determine the fragrance oil mass loss results at the end. Fan speed was set to 1700 RPM (60 CFM) and rotating disc speed was 7.5 rpm, with the edge of the device placed at the edge of the disc (22 cm from the center).

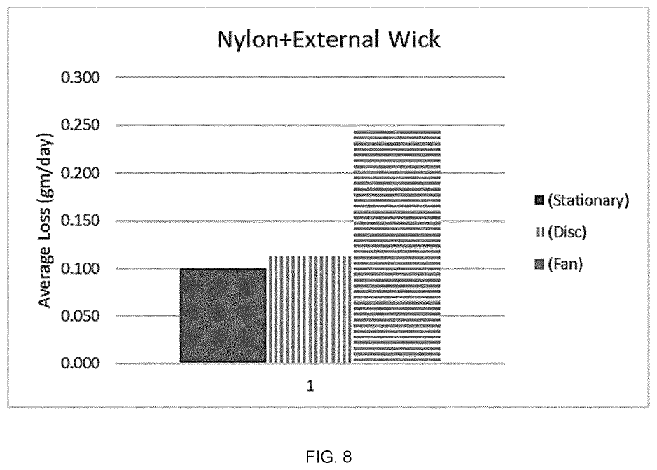

FIG. 8 shows the amount of mass loss (in grams per day) under the varying conditions. As shown in FIG. 8, the mass loss varies based on the condition scaffolds were exposed to. The stationary state showed the lowest fragrance loss, followed by rotating disc and fan giving the highest fragrance loss.

### Example 6. Mass Loss of Another Exemplary Device with External Wick and Reservoir Under Various Conditions

Scaffold F according to Example 3 (polypropylene scaffold with external wick) was subjected to various conditions: stationary state, positioned on a rotating disc, and positioned under a fan to evaluate fragrance loss. Scaffold F was placed at a fixed position on a rotating disc platform and mass loss amount was noted while alternating on a regular basis over several days the following conditions: stationary state, rotating disc state and fan state. The scaffold was pre-weighed to accurately determine the fragrance oil mass loss results at the end. Fan speed was set to 1700 RPM (60 CFM) and rotating disc speed was 7.5 rpm, with the edge of the device placed at the edge of the disc (22 cm from the center).

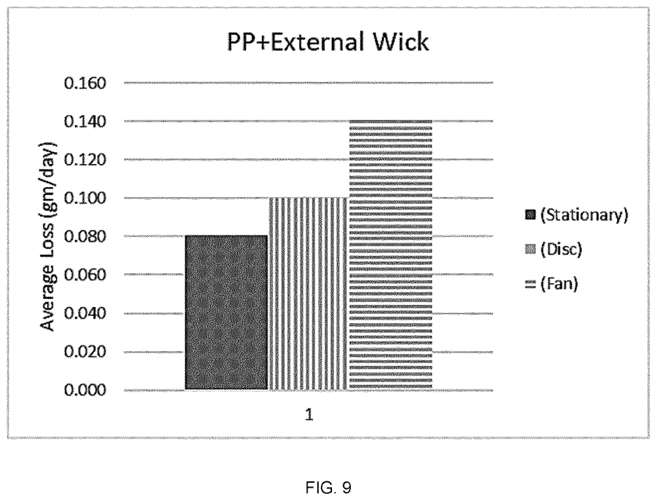

FIG. 9 shows the amount of mass loss (in grams per day) under the varying conditions. As shown in FIG. 9, the mass loss varies based on the condition scaffolds were exposed to. The stationary state showed the lowest fragrance loss, followed by rotating disc and fan giving the highest fragrance loss.

### Example 7. Exemplary Device Having a Dual Reservoir and Double Gyroid Scaffold

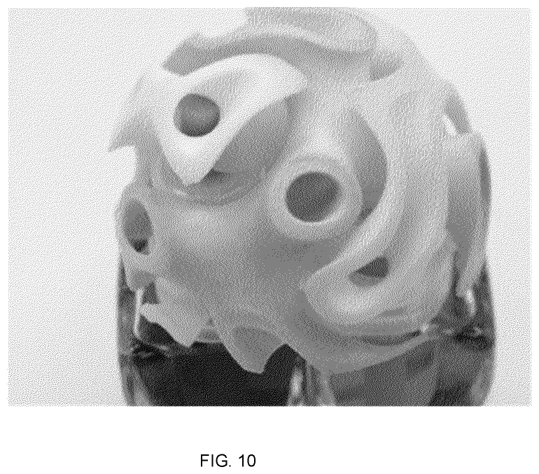

A dual release fragrance delivery device featuring a double gyroid structure (“Sample A1”) in which two reservoirs were connected to separate intertwined emanating surfaces through separate wicks was made. The fragrance oil was a fragrance of a fruity/floral type, with green and yellow color added to show the migration of fragrance to the intertwined structures. The scaffold structure held about 2.7 wt % of fragrance when fully saturated. FIG. 10 shows the exemplary device having a dual reservoir and double gyroid scaffold.

### Example 8. Exemplary device having an embedded reservoir

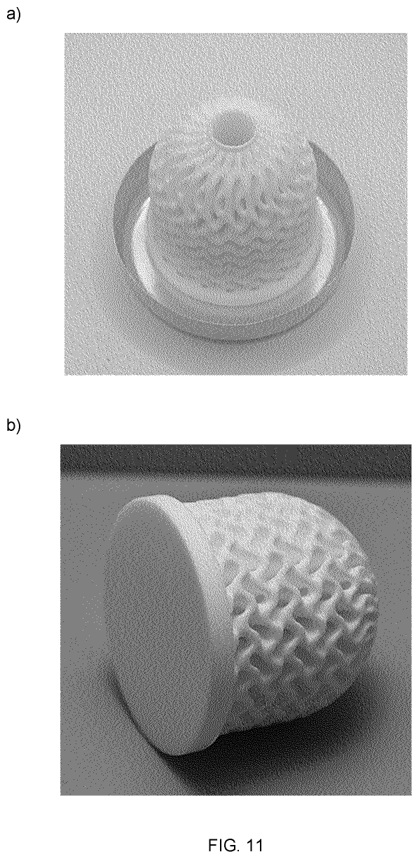

A fragrance delivery device featuring an embedded reservoir (“Sample B”) was made with a field-driven design in which the pore size was varied radially. As shown in FIGS. 11a and 11b, the fragrance delivery device features a central reservoir that can be refilled using any dispensing device, such as a pipette. The central cavity is dense enough to hold the liquid, yet porous enough to allow the fragrance oil to penetrate and diffuse radially. It performs as an aesthetic and performant air freshener that can easily be refilled when depleted.

### Example 9. Exemplary Devices Each Having a Single Reservoir

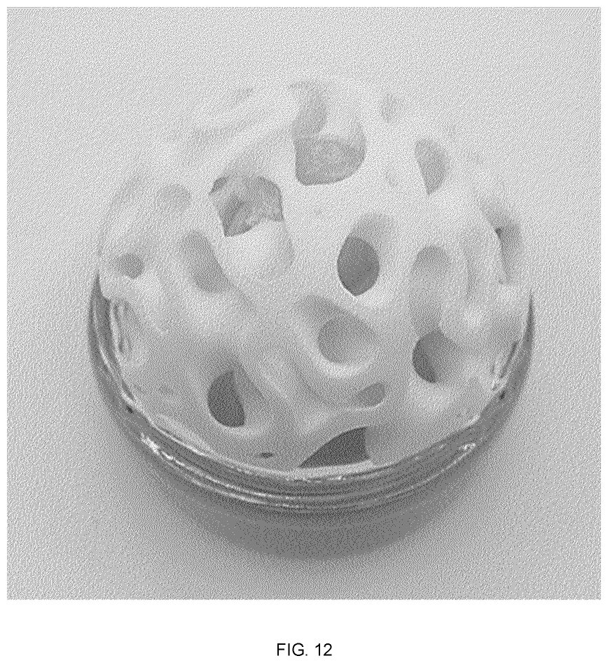

A fragrance delivery device having a single reservoir (“Sample C1”) was constructed from a commercially available air freshener bottle and central wick with a yellow-colored fragrance oil. A scaffold according to the present disclosure was designed to fit perfectly with the wick and glass reservoir to ensure the two were fluidly connected. It was clear from the color transfer that the liquid can move easily towards the surface of object. The scaffold held about 2 wt % of fragrance when fully saturated. FIG. 12 shows the exemplary fragrance delivery device having a single reservoir.

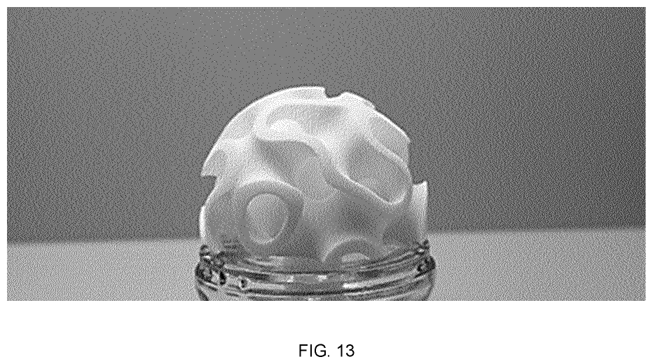

Another fragrance delivery device having a single reservoir (“Sample D1”) was similarly constructed with a central wick and reservoir, but with a different scaffold according to the present disclosure. FIG. 13 shows another exemplary fragrance delivery device having a single reservoir.

### Example 10. Exemplary Devices Having Ceramic Scaffolds

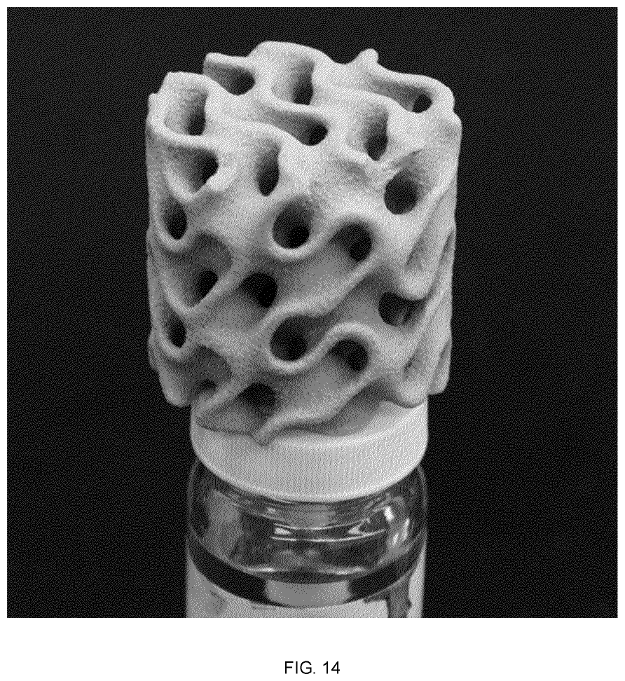

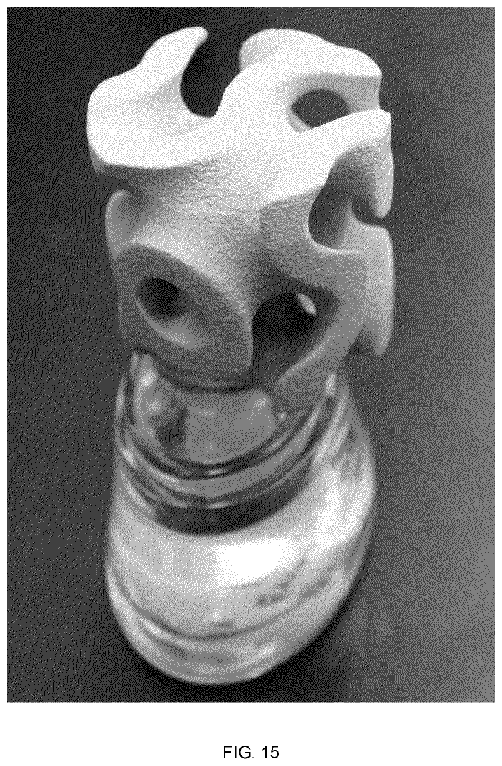

Fragrance delivery devices having ceramic scaffolds (“Sample E1” and “Sample E2”) were constructed. Traditional wicks were used to connect the scaffolds to a reservoir of fragrance. The ceramic scaffolds have high porosity and are capable of holding about 31-35% of their own weight in fragrance. FIG. 14 shows exemplary fragrance delivery device Sample E1 and FIG. 15 shows the exemplary fragrance delivery device Sample E2.

### Example 11. Mass Loss Experiments on Exemplary Devices

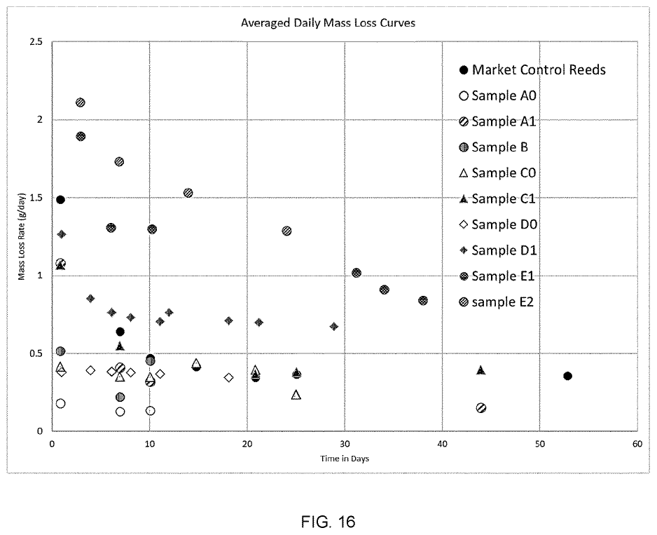

The exemplary fragrance devices described in Examples 7 to 10 were subjected to mass loss studies. The exemplary fragrance devices described in Examples 7 to 10 were positioned in a specialized room, with temperature and humidity control that was maintained at a temperature of ~ 21.5° C. and 50% relative humidity. Weight loss was monitored over time. Sample A0, Sample C0, and Sample D0, which correspond to Sample A1, Sample C1, and Sample D1, respectively, but without the scaffolds according to the present disclosure, were used as controls. Market reed air fresheners were also used as a control. FIG. 16 shows the average daily mass loss rate vs time of the exemplary fragrance devices and controls.

As shown in FIG. 16, the control samples without scaffolds, designated by ending in suffix ‘0’, exhibited relatively poor performance. Sample B performed well, but depleted quickly and had to be refilled after a week so that it could regain its initial rate of mass loss at time=10 days. Other scaffolds according to some aspects of the present disclosure clearly improved the performance against their controls (compare A1 vs A0; C1 vs C0; D1 vs D0). Some of these were on-par with the market standard of reed air fresheners, but some even clearly outperformed these, see sample D1, E1 and E2. The samples that performed the best were the high-porosity ceramic devices, Samples E1 & E2, which easily outperformed the Market control by 2-3 fold between ~1 to 5 weeks.

### Example 12. Shell SLS 3D Printing and Comparison with Normal SLS 3D Printing

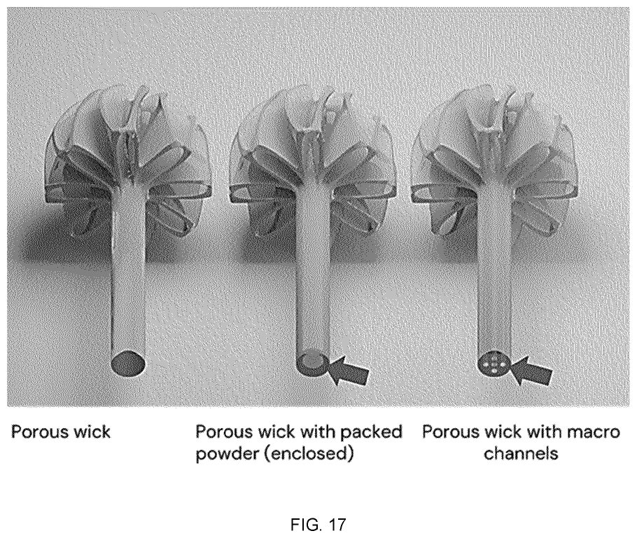

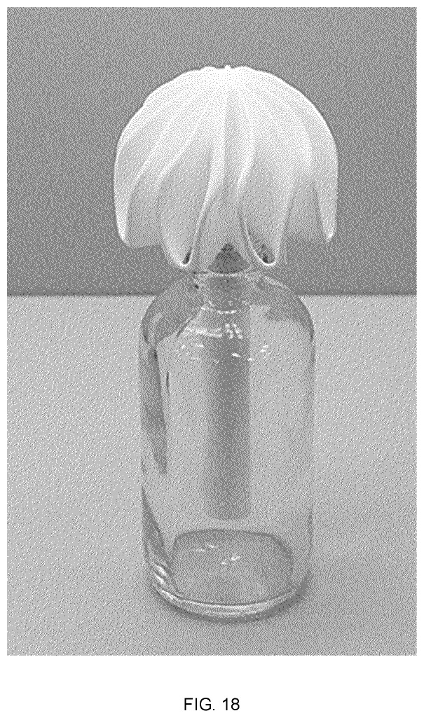

Three variations of a scaffold having a triply periodic minimal surface geometry with a connected wick were produced. The three variations varied in the wick portion. In the first variation (“Sample F1”), the wick portion was a porous wick. In the second variation (“Sample F2”), the wick portion was a porous wick with packed powder. In the third variation (“Sample F3”), the wick portion was a porous wick with macro channels. These variations were produced using both shell and normal SLS 3D printing. FIG. 17 shows the three variations of a scaffold having a triply periodic minimal surface geometry with a connected wick. FIG. 18 shows a variation of the scaffold having a triply periodic minimal surface geometry with a connected wick and a reservoir.

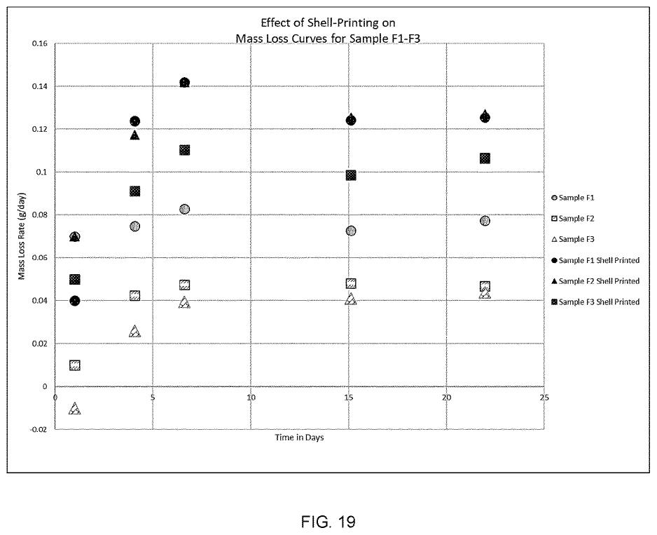

The shell printed and normally printed scaffolds with connected wicks were positioned in a bottle containing fragrance, whereby the wick portion was used to transport fragrance oil to the emanating surface. Weight loss was monitored in a specialized room, with temperature and humidity control that was maintained at a temperature of ~ 21.5° C. and 50% relative humidity. Weight loss was monitored over time. FIG. 19 shows the average daily mass loss rate vs time of the exemplary fragrance devices with shell printed and normally printed scaffolds and connected wicks.

The results show that this technique of shell-printing can improve the performance since all of the fragrance delivery devices having the shell-printed variations outperformed the regularly printed counterparts, almost doubling the mass loss rate.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A device comprising:
a) a body portion comprising a porous material,
wherein the body portion has a volume and at least one surface,
wherein the volume comprises at least one network of a plurality of fluidly connected passages,
wherein the at least one network of fluidly connected passages has at least one first end and at least one second end,
wherein the at least one first end and at least one second end are separated by a distance,
wherein at least one of the first or second ends are fluidly connected to the at least one surface,
wherein each individual passage within the plurality has a cross section,
wherein the distance, and the cross section of each passage within the plurality defines a surface, and
b) at least one reservoir comprising a liquid fragrance composition fluidly connected to the body portion;
wherein the fluid connection is configured to draw the liquid fragrance composition into the porous material of the body portion,
wherein the porous material of the body portion is configured to absorb the liquid fragrance composition, and
wherein the surface of the body portion is configured to disperse the liquid fragrance composition via evaporation.

2. The device of claim 1, wherein each individual passage within the plurality has one or more branches.

3. The device of claim 1, wherein the surface comprises a triply periodic minimal surface geometry.

4. The device of claim 1, wherein the triply periodic minimal surface geometry is selected from the group consisting of: a gyroid geometry, a lidinoid geometry, a Schwarz D “diamond” geometry, or a Schwarz P “primitive” structure geometry.

5.-7. (canceled)

8. The device of claim 1, wherein the cross section of each individual passage within the plurality varies.

9. The device of claim 8, wherein the cross section of each individual passage within the plurality is greater in the center of the body portion than the cross section of each individual passage within the plurality at the periphery of the body portion.

10. The device of claim 8, wherein the cross section of each individual passage within the plurality is greater at the periphery of the body portion than the cross section of each individual passage within the plurality at the center of the body portion.

11. The device of claim 1, wherein the cross section of each individual passage within the plurality is at least 1 mm, typically at least 5 mm, more typically at least 10 mm.

12. (canceled)

13. (canceled)

14. The device of claim 1, wherein the body portion has an open pore porosity of 0.01 to 0.9.

15. The device of claim 14, wherein the body portion comprises a plurality of pores, the pores having a size of less than 1,000 μm.

16. The device of claim 1, wherein the body portion comprises porous porcelain materials, plastics, molded ceramics, glass fibers, clay, activated carbon, cellulose, wood materials, and any combination thereof.

17. The device of claim 16, wherein the body portion comprises nylon, polypropylene, or a combination thereof.

18. The device of claim 1, wherein the at least one reservoir comprising the liquid fragrance composition is fluidly connected to the body portion through at least one wick.

19. The device of claim 18, wherein the at least one wick comprises a porous material.

20. The device of claim 1, wherein the liquid fragrance composition has a density of from about 0.7 to about 1.3 g/mL.

21. The device of claim 1, wherein the liquid fragrance composition has a surface tension of about 10 to about 70 mN/m.

22. The device of claim 1, wherein the liquid fragrance composition has a viscosity of about 0.1 to about 10,000 mPa·s.

23. (canceled)

24. A method of dispensing a liquid fragrance composition into a surrounding space, comprising placing the device of claim 1 into a space in need thereof, and allowing the liquid fragrance composition to evaporate from the device.

25. A kit, comprising:
a) a body portion comprising a porous material,
wherein the body portion has a volume and at least one surface,
wherein the volume comprises at least one network of a plurality of fluidly connected passages,
wherein the at least one network of fluidly connected passages has at least one first end and at least one second end,
wherein the at least one first end and at least one second end are separated by a distance,
wherein at least one of the first or second ends are fluidly connected to the at least one surface,
wherein each individual passage within the plurality has a cross section,
wherein the distance, and the cross section of each passage within the plurality defines a surface, and
b) at least one reservoir comprising a liquid fragrance composition.

26. The kit of claim 25, further comprising at least one wick configured to fluidly connect the at least one reservoir with the body portion.
